(19) United States
(12) Patent Application Publication
(10) Pub. No.: US 2026/0108573 A1
(43) Pub. Date: Apr. 23, 2026

(54) CALLUNA VULGARIS FOR TREATING AND PREVENTING URINARY TRACT INFECTIONS

(71) Applicant: Desert Harvest, Inc., Ellsworth, ME (US)

(72) Inventor: Heather Florio, Brooksville, ME (US)

(21) Appl. No.: 19/322,118

(22) Filed: Sep. 8, 2025

Related U.S. Application Data
(60) Provisional application No. 63/710,708, filed on Oct. 23, 2024.

(51) Int. Cl. A61K 36/45; A61K 9/14; A61P 13/02

(52) U.S. Cl. CPC A61K 36/45; A61K 9/14; A61P 13/02

(57) ABSTRACT

Provided herein are methods and compositions of Calluna vulgaris for the prevention and treatment of Urinary Tract Infections.

### CROSS-REFERENCE TO RELATED APPLICATIONS

The present application claims priority to U.S. Provisional Patent Application No. 63/710,708, filed Oct. 23, 2024, herein incorporated by reference in its entirety.

### BACKGROUND

The invention generally relates to compositions and treatments for urinary tract infections (UTI's).

Recurrent UTI's are defined by the American Family Physician Association, as UTI's that occur more than twice in 6 months or more than three times in a 12-month period. Currently, the standard over-the-counter recommendation by doctors is D-mannose, which in research has only shown to bind to E. coli bacteria. In a 2014 study in Bosnia, Calluna vulgaris (Heather) was shown to successfully bind to three different types of bacteria, including Escherichia coli, Enterococcus faecalis and Proteus vulgaris. D-mannose has been shown to cause diarrhea and nausea for people that take it, with the long-term effects unknown.

The present invention attempts to solve these problems as well as others.

### SUMMARY OF THE INVENTION

Provided herein are methods and compositions of Calluna vulgaris for the prevention and treatment of Urinary Tract Infections.

The methods and compositions are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the FIGURES, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF EMBODIMENT

Generally speaking, the present invention comprises a composition or formulation of an active ingredient or pharmaceutical composition from Calluna vulgaris ((C. vulgaris) for the prevention and treatment of Urinary Tract Infections. In one embodiment, the active ingredient or pharmaceutical composition from Calluna vulgaris (Heather) plant is in powder or tincture form for the prevention and treatment of UTIs. This Calluna vulgaris composition or formulation replaces D-mannose as a more effective preventative of recurrent UTI's.

In one embodiment, delivery method studies are conducted to determine the most effective delivery method: tincture or powder in capsule. In one embodiment, efficacy studies provide the best delivery method to treat and/or prevent UTIs.

The Calluna vulgaris composition or formulation comprises an effective amount of phenols or including a phenolic content between about 67.55 mg to about 142.46 mg of GAE/g of Calluna Vulgaris extract and an effective amount of flavonoid content between about 42.11 to about 63.68 mg RUE/g of Calluna vulgaris extract.

The Calluna vulgaris composition or formulation comprises an antibacterial activity between about 2.5 Minimum inhibitory concentration (MIC) and 40 MIC and an antibacterial activity between about 2.5 minimum bactericidal concentration (MBC) and about 40 MBC.

In some embodiments, a Calluna vulgaris formulation provided herein may include 0.1 mg/ml to 40 mg/ml of Calluna vulgaris. In some embodiments, the Calluna vulgaris formulation may be admixtures of bioactives. For example, a Calluna vulgaris formulation provided herein may comprise 0.1 mg/ml to 30 mg/ml, 0.5 mg/ml to 30 mg/ml, 1 mg/ml to 30 mg/ml, 0.1 mg/ml to 25 mg/ml, 0.5 mg/ml to 25 mg/ml, 1 mg/ml to 25 mg/ml, 0.1 mg/ml to 20 mg/ml, 0.5 mg/ml to 20 mg/ml, 1 mg/ml to 20 mg/ml, 0.1 mg/ml to 15 mg/ml, 0.5 mg/ml to 15 mg/ml, 1 mg/ml to 15 mg/ml, 0.1 mg/ml to 10 mg/ml, 0.5 mg/ml to 10 mg/ml, 1 mg/ml to 10 mg/ml, 0.1 mg/ml to 5 mg/ml, 0.5 mg/ml to 5 mg/ml, 1 mg/ml to 5 mg/ml, 0.1 mg/ml to 2 mg/ml, 0.5 mg/ml to 2 mg/ml, 1 mg/ml to 2 mg/ml, 2 mg/ml to 40 mg/ml, 2 mg/ml to 30 mg/ml, 2 mg/ml to 25 mg/ml, 2 mg/ml to 20 mg/ml, 2 mg/ml to 15 mg/ml, 2 mg/ml to 10 mg/ml, 2 mg/ml to 5 mg/ml, 5 mg/ml to 40 mg/ml, 5 mg/ml to 30 mg/ml, 5 mg/ml to 25 mg/ml, 5 mg/ml to 20 mg/ml, 5 mg/ml to 15 mg/ml, 5 mg/ml to 10 mg/ml, 10 mg/ml to 40 mg/ml, 10 mg/ml to 30 mg/ml, 10 mg/ml to 25 mg/ml, 10 mg/ml to 20 mg/ml, 10 mg/ml to 15 mg/ml, 15 mg/ml to 40 mg/ml, 15 mg/ml to 30 mg/ml, 15 mg/ml to 25 mg/ml, 15 mg/ml to 20 mg/ml, 20 mg/ml to 40 mg/ml, 20 mg/ml to 30 mg/ml, 20 mg/ml to 25 mg/ml, 25 mg/ml to 40 mg/ml, 25 mg/ml to 30 mg/ml, 30 mg/ml to 40 mg/ml, or between 5 mg/ml and 125 mg/ml of Calluna vulgaris.

In some embodiments, a Calluna vulgaris formulation provided herein may comprise at least 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, or between 0.45 mg/ml to 4.5 mg/ml of Calluna vulgaris.

In some embodiments, a Calluna vulgaris formulation provided herein may comprise 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, 40 mg/ml, or between 50 mg/ml and 5000 mg/ml of Calluna vulgaris.

In some embodiments, a Calluna vulgaris formulation provided herein may comprise 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, or between 400 mg/ml and 4000 mg/ml of Calluna vulgaris.

In another aspect, the present invention encompasses a method for treating an UTI condition or disorder in a mammalian subject (preferably, but not exclusively, a mammalian subject) in need of such treatment, wherein said method comprises the administration (systemically, topically or by a combination of routes) of a composition of the present invention.

The present invention further provides a Calluna vulgaris formulation for use as medicament or other therapeutic entity (such as ‘herbal remedy’, ‘food supplement’ and the like) in the treatment of an inflammatory condition. In one embodiment of this aspect, said composition is provided for use as a medicament or other therapeutic entity in the treatment of a systemic or topical or mucosal UTI.

The present invention further provides the use of a Calluna vulgaris formulation as disclosed herein for the preparation of a medicament. In some embodiments, this aspect of the invention relates to the use of a conjugated formulation as disclosed herein in the preparation of a medicament for use in the treatment of an inflammatory condition or disorder.

In certain embodiments, the dosage form is formulated as granules, pellets, micro particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, toffee, chocolate or cookie. In certain embodiments, the tablet or pellets are an immediate release or slow or controlled release dosage forms. In certain embodiments, the tablet is enteric coated or is a melt or dissolved in the mouth or is muco-adhesive dosage form.

In certain embodiments, the unit dosage form which is a unit particles, such as tablet, capsule, granules, pellets, micro-particles and film, are enteric coated or coated with a colonic coat that protect the unit dose from being decomposed at the acidic gastric pH and swells in time manner of pH controlled manner or both, to release the cannabinoids at the distal intestine and may also release part of the cannabinoids in the intestines for systemic absorption and part of the Calluna vulgaris at the urinary tract for local urinary tract pharmacological effect.

In certain embodiments, the Calluna vulgaris formulation is formulated in a semi solid or liquid dosage form such as cream, lotion, ointment, dispersion, suspension, gel, foam, spray, syrup, liquid, eye drops, ear drops, enema or an oral dosage form or a topical dosage form or a local ophthalmic or optic or oral cavity or vaginal or rectal or uterine dosage form.

In certain embodiments, any one of the compositions described above, or any one of the dosage forms described above, is for use in a method of treating UTI symptoms or disorders. Preferred dosage forms include, but are not limited to, any liquid or semi solid or solid dosage form. The composition may be formulated in a medicament by preparing a topical or mucosal or oral delivery system. The topical delivery system may be in form of eye drops, a suspension, ointment, cream, foam, spray, topical patch. The oral delivery system may be a tablet or capsule or soft capsule or sachet or granules or a syrup. The mucosal delivery system may be a gel, pessary, enema, douche, wash, foam, mucoadhesive gel or tablet for immediate or for slow or controlled release. The vehicle may comprise any acceptable solvent and inactive ingredients as well as preservatives anti-oxidants and coloring agents. The delivery form may be single dose or multiple dose as well as micro particle granulate nanoparticle microcapsule liposome micelle, and the like as known in the art of pharmaceutical, cosmetic, veterinary medicine and art of formulation. Further details of suitable dosage forms may be obtained from any standard reference work in this field, including, for example: Remington's Pharmaceutical Sciences, Mack Publishing Co, Easton, Pa, USA (1980).

Thus, in some embodiments of the present invention, the composition further comprises one or more excipients selected from the group consisting of solvents, stabilizers, suspending agents, emulsifiers, release modifying, targeting and viscosity agents and combinations thereof.

In some embodiments, the composition of the present invention is formulated as a dosage form selected from the group consisting of a liquid, a suspension, an emulsion, a foam, a spray, a liposome, a semi-solid, a cream, an ointment, a patch, a particulate formulation, a granulate, a micro-particulate formulation, a nano-particulate formulation, a solid dosage form, a tablet, a capsule, an orally-disintegrable capsule, a mouth wash and an adhesive buccal tablet.

According to particular embodiments, the compounds or derivatives prepared according to embodiments of the methods of the present disclosure can comprise compounds or derivatives, or salts, hydrates, solvates, or prodrugs thereof, or crystalline forms thereof, substantially free of solvents or other by-products, generally, or a particular solvent or by-product. In certain embodiments, by “substantially free” is meant greater than about 80% free of solvents or by-products, or greater than about 80% free of a particular solvent or by-product, more preferably greater than about 90% free of solvents or by-products, or greater than about 90% free of a particular solvent or by-product, even more preferably greater than about 95% free of solvents or by-products, or greater than about 95% free of a particular solvent or by-product, even more preferably greater than 98% free of solvents or by-products, or greater than about 98% free of a particular solvent or by-product, even more preferably greater than about 99% free of solvents or by-products, or greater than about 99% free of a particular solvent or by-product, even more preferably greater than about 99.99% free of solvents or by-products, or greater than about 99.99% free of a particular solvent or by-product, and most preferably quantitatively free of solvents or by-products, or quantitatively free of a particular solvent or by-product.

For preparing pharmaceutical compositions from a Calluna vulgaris form or a hydrate, solvate, or prodrug thereof, prepared according to the methods of the present disclosure, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active components. In tablets, the active component is mixed with the carrier having the necessary binding capacity in suitable proportions and compacted in the shape and size desired.

The powders and tablets preferably contain from about 1 to about 99.99 percent of the active form of Calluna vulgaris, or salt, hydrate, solvate, or prodrug thereof, prepared according to the methods of the present disclosure. Suitable carriers are microcrystalline cellulose, sugar, lactose, pectin, dextrin, starch, gelatin, tragacanth, methylcellulose, sodium carboxymethylcellulose, a low melting wax, cocoa butter, and the like, and other excipients may include magnesium stearate, stearic acid, talc, silicon dioxide, etc. Dosages of the active form of Calluna vulgaris may be between about 10 mg to about 10000 mg in the preparation for example. The term “preparation” is intended to include the formulation of active compound with encapsulating material as carrier providing a capsule in which the active component, with or without carriers, is surrounded by a carrier, which is thus in association with it. Tablets, powders, capsules, pills, sachets, and lozenges are included. Tablets, powders, capsules, pills, sachets, and lozenges can be used as solid forms suitable for oral administration. Liquid preparations include solutions, suspensions, and emulsions, for example, water or water-propylene glycol solutions. For example, parenteral injection liquid preparations can be formulated as solutions in aqueous polyethylene glycol solution. The crystalline forms of Calluna vulgaris extracts, or salts, hydrates, solvates, or prodrugs thereof prepared according to the methods of the present disclosure may thus be formulated for parenteral administration (e.g., by injection, for example bolus injection or continuous infusion) and may be presented in unit dose for example in ampoules, pre-filled syringes, small volume infusion, or in multi-dose containers with an added preservative). The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use. The method of administration may be via inhalation and topical routes. Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration in the urinary tract include the active ingredient in an inert base such as gelatin and glycerine or sucrose and acacia; and mouthwashes comprising the active ingredient in suitable liquid carrier.

Solutions or suspensions are applied directly to the urinary tract by conventional means, for example with a dropper, pipette, or spray. The compositions may be provided in single or multi-dose form. In compositions intended for administration to the respiratory tract, including intranasal compositions, the compound or derivative will generally have a small particle size, for example on the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets, capsules, tinctures, and lozenges for oral administration and liquids for oral use are preferred compositions. Solutions or suspensions for application to the nasal cavity or to the respiratory tract are preferred compositions. Transdermal patches for topical administration to the epidermis are preferred compositions.

Further details on techniques for formulation and administration may be found in the latest edition of REMINGTON'S PHARMACEUTICAL SCIENCES (Mack Publishing Co., Easton, PA).

Aqueous solutions suitable for oral use can be prepared by dissolving the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Further details on techniques for formulation may be found in the latest edition of Remington's Pharmaceutical Sciences (Mack Publishing Co., Easton, PA).

### Definitions

The term “administration” or “administering” refers to a method of providing a dosage of a compound or active ingredient or pharmaceutical composition to a subject, where the method is epicutaneous (topical) or subcutaneous. Modes of administration, dosing schedules disclosed compounds and compositions can be determined according to the criteria generally taken into account in the establishment of a UTI treatment adapted to, for example, a patient's urinary tract. The compositions can be administered such that they cover the entire area to be treated.

In the present disclosure, an “effective amount” or an “effective dose” of C. vulgaris, or composition refers to an amount of (C. vulgaris that, once administered to a subject, will reach the subject's bloodstream and/or bodily tissues.

The term “pharmaceutical composition” as used herein has its conventional meaning and refers to a composition which is pharmaceutically acceptable. The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. Pharmaceutical composition includes configurational isomers (such as cis and trans isomers) and all optical isomers (such as enantiomers) Isomers and diastereomers), racemic, diastereoisomers and other mixtures of these isomers, as well as solvates, hydrates, isomorphs, polymorphs, tautomers, Ester, salt forms and prodrugs. The term “prodrug” refers to a compound that is a drug precursor, which releases the drug in vivo through some chemical or physiological processes after administration (for example, the prodrug is transformed into the desired drug form when it reaches physiological pH or through the action of enzymes). Exemplary prodrugs release the corresponding free acid upon cleavage, and the hydrolyzable ester-forming residues of the compounds of the present invention.

The term “pharmaceutically acceptable” refers to derivatives, analogues and salts which are physiologically acceptable for use in mammals, and which are not unduly toxic or otherwise unacceptable for such use. The term “mammals” includes human and non-human mammals, including domestic animals, e.g. cats, dogs, rodents, cattle, horses and the like, as well as non-domesticated animals.

The term “excipient” as used herein has its conventional meaning and refers to a pharmaceutically acceptable ingredient, which is commonly used in the pharmaceutical technology for preparing a granulate, solid or liquid oral dosage formulation. The term “cosmetic composition” is intended to mean a substance or a preparation intended to be brought into contact with the various superficial parts of the body, in particular the epidermis, the body-hair and head-hair systems, the nails, the lips and the oral mucous membranes. The term “veterinary composition” encompasses the full range of compositions for internal administration and feeds and drinks which can be consumed by animals.

As used herein, the term “solvent” refers to a compound or mixture of compounds including, but not limited to, water, water in which an ionic compound has been dissolved, acetic acid, acetone, acetonitrile, benzene, 1-butanol, 2-butanol, t-butyl alcohol (“TBA”), 2-butanone, carbon tetrachloride, chlorobenzene, chloroform, cyclohexane, 1,2-dichloroethane (“DCE”), diethylene glycol, diethyl ether (“Et₂O”), diglyme (diethylene glycol dimethyl ether), 1,2-dimethoxyethane (“DME”), N,N-dimethylformamide (“DMF”), dimethylsulfoxide (“DMSO”), 1,4-dioxane, ethanol, ethyl acetate (“EtOAc”), ethylene glycol, glycerin, heptanes, hexamethylphosphoramide (“HMPA”), hexamethylphosphorus triamide (“HMPT”), hexane, methanol (“MeOH”), methyl 1-butyl ether (“MTBE”), methylene chloride (“DCM,” “CH₂Cl₂”), N-methyl-2-pyrrolidinone (“NMP”), nitromethane, pentane, petroleum ether, 1-propanol (“n-propanol,” “n-PrOH”), 2-propanol (“isopropanol,” “iPrOH”), pyridine, tetrahydrofuran (“THF”), toluene, tributylamine, triethylamine (“TEA,” “Et₃N”), o-xylene, m-xylene, and/or p-xylene, and the like. Solvent classes may include hydrocarbon, aromatic, aprotic, polar, alcoholic and mixtures thereof.

The term “synergistic” as used herein is refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-inflammatory; ingredient B is pro-inflammatory; when A and B are combined, said combination produces an anti-inflammatory effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Preparation of Plant Extract

Prepared plant material (10 g) was transferred to dark-colored flasks with 200 ml of solvent (water, ethanol, ethyl acetate) and stored at room temperature. After 24 h, infusions were filtered through Whatman No. 1 filter paper and residue was re-extracted with equal volume of solvents. After 48 hours, the process was repeated. Combined supernatants were evaporated to dryness under vacuum at 40° C. using Rotary evaporator. Aqueous extract of herb was prepared at 80° C. The obtained extracts were kept in sterile sample tubes and stored at −20° C.

### Determination of Total Phenolic Content

The C. vulgaris extracts were analyzed by spectrophotometry for total phenolics according to Folin-Ciocalteu procedure [13]. The reaction mixture was prepared by mixing 0.2 ml of methanolic solution of extract (1 mg/ml) and 1.5 ml of 110 Folin-Ciocalteu reagent dissolved in water. The mixture was allowed to equilibrate for 5 min and then mixed with 1.5 ml 6 NaCO₃ solution. After incubation for 90 minutes at room temperature in darkness, the absorbance of the mixture was read at 725 nm against a blank using spectrophotometer. The blank was prepared with methanol instead of extract solution. The samples were prepared in triplicate and the mean value of absorbance was obtained. The same procedure was repeated for gallic acid, which was used for calibration of standard curve. Total phenol content is reported as gallic acid equivalents by reference to linear equation of the standard curve (y=0.008x+0.0077, R²=0.998). Then the total phenolic content was expressed as gallic acid equivalents in milligrams per gram of extract (mg GAE/g of extract).

### Determination of Flavonoid Concentration

The concentrations of flavonoids were determined using spectrophotometric method with aluminum chloride [14]. The sample contained 1 ml of methanolic solution of the extract in the concentration of 1 mg/ml and 1 ml of 2 AlCl₃ solution dissolved in methanol. The mixture was vigorously shaken, and after 10 minutes of incubation at room temperature, the absorbance versus a prepared blank was read at 430 nm using spectrophotometer. The samples were prepared in triplicate and the mean value of absorbance was obtained. Rutin was used as a standard for calibration of standard curve. The concentrations of flavonoids were calculated from the linear equation of standard curve (y=0.021x+0.040, R²=0.999). Then the concentrations of flavonoids were expressed as milligram of rutin equivalent per gram of extract (mg of RUE/g of extract).

### Tested Bacterial Strains

Antibacterial activity of aqueous, ethanol and ethyl acetate extract from C. vulgaris was tested against urinary pathogens, extracted from urine samples, including ten different Escherichia coli strains (Mf-Ec1, Mf-Ec2, Mf-Ec3, Mf-Ec4, Mf-Ec5, Mf-Ec6, Mf-Ec7, Mf-Ec8, Mf-Ec9, Mf-Ec10), ten Enterococcus faecalis strains (Mf-Ef1, Mf-Ef2, Mf-Ef3, Mf-Ef4, Mf-Ef5, Mf-Ef6, Mf-Ef7, Mf-Ef8, Mf-Ef9, Mf-Ef10) and ten different strains of Proteus vulgaris (Mf-Pv1, Mf-Pv2, Mf-Pv3, Mf-Pv4, Mf-PvS, Mf-Pv6, Mf-Pv7. Mf-Pv8, Mf-Pv9, Mf-Pv10).

The Escherichia coli strains and Proteus vulgaris strains represented Gram-negative bacteria. Bacterial strains of Enterococcus faecalis were Gram-positive. These pathogens are the most frequent cause of urinary tract infections [15].

### Suspension Preparation

The original density of the bacterial suspension was 0.5 Mc Farland after which the additional dilution in saline at the pro-portion of 110 is made. The final concentration of the bacteria in the test tubes was 10⁶ colony forming units (CFU)/ml.

### Microdilution Method

The minimum inhibitory concentration (MIC) of the extracts had been determined using the tube dilution method through the series of dilutions [16]. In the test tubes filled with the Mueller Hinton broth, the solution of the extracts is added and the series of double dilutes was made. In each of the test tubes 100 μl of the suspension of the tested bacteria was added. The mixture was incubated for 24 hours at the temperature of 37° C. The same method was used to identify the MIC value for amoxicillin. These values have been determined by inoculating the Mueller Hinton agar with the test tube content. Amoxicillin was used as a positive control. Whereas the extracts were dissolved in 10% DMSO, solvent control test was performed to study the effects of 10% DMSO on the growth of bacterial strains. It was observed that 10% DMSO did not inhibit the growth of bacteria.

### Statistical Analysis

Data are presented as means±standard deviations where appropriate. All statistical analyses were performed using SPSS package (IBM Corp. Chicago, USA).

### Results

### Total Phenolic Content and Flavonoid Concentrations

The results of total phenolic content and flavonoid concentrations in tested extracts from C. vulgaris are presented in Table 1. The total phenolic content was expressed as gallic acid equivalents. The aqueous extract had the highest phenolic content with 142.46 mg of GAE/g of extract. The ethanol extract was richer in phenolic active compounds than ethyl acetate extract. The content of flavonoids was expressed as rutin equivalent. Total flavonoid content in plant extracts ranged between 42.11 to 63.68 mg RUE/g of extract. High concentration of flavonoids was measured in ethyl acetate extract from C. vulgaris.

### Antibacterial Activity

In vitro antibacterial activities of aqueous, ethanol and ethyl acetate extracts of C. vulgaris leaves and flowers were studied on strains of Gram-positive and Gram-negative bacteria. The results of antibacterial activities of extracts against 30 strains of pathogenic bacteria are presented in Table 2. Antibacterial activity of tested extracts was evaluated by determining MICs and MBCs values. All tested extracts from C. vulgaris inhibited urinary pathogens extracted from urine samples.

TABLE 1
Total phenolic contents and concentrations of flavonoids
in leaves and flowers of C. vulgaris extracts

| | Total phenolic content¹ | Flavonoid concentration¹ |
|---|---|---|
| Type of extract | (mg GAE/g of extract) | (mg RUE/g of extract) |
| Water | 142.46 ± 0.50 | 42.11 ± 0.32 |
| Ethanol | 81.86 ± 0.95 | 43.03 ± 0.11 |
| Ethyl acetate | 67.55 ± 0.38 | 63.68 ± 0.19 |

The strongest antibacterial activity was detected on P. vulgaris while the activities on E. coli and E. faecalis strains were similar. Aqueous extract showed the strongest effect against all tested bacterial strains. In general, the tested extracts demonstrated selective antibacterial activity and the activity depended both on the species of bacteria and on the type of extract. Ethyl acetate extract showed excellent activity on strains of P. vulgaris.

### CONCLUSIONS

Previous research has indicated that phenolic compounds have antibacterial properties [17].

Phenolics belong to the group of secondary plant com-pounds and have various bioactivities such as antioxidat [18], anti-ulcer [19], and anti-inflammatory [20]. The interest in possible health benefits of flavonoids has increased due to their powerful antimicrobial activities [21].

Extract of C. vulgaris was found effective against M. tuberculosis bacillus and the results of this study provide scientific basis for the traditional use of this plant in the treatment of tuberculosis [22].

Huttunen et al. [23] investigated antimicrobial activity of five Finnish honey products against important human pathogens Streptococcus pneumoniae, S. pyogenes and Staphylococcus aureus. In this research, heather (C. vulgaris) honey showed significant antimicrobial activity against all tested pathogens. Heather honey was tested against Staphylococcus aureus and C. vulgaris was shown it could be a source that could provide honey with high antibacterial activity [24].

Previous studies have shown that species belonging to the family Ericaceae are rich in phenolics [25,26]. The anti-microbial activity of phenolic compounds from heather is also significant. Antibacterial effects of different extracts of C. vulgaris showed that phenolic compounds and flavonoids were responsible for the growth inhibition of bacterial strains.

This study indicated that extracts of C. vulgaris have anti-bacterial activity and may have potential medical use. Studies of medicinal plants are important sources of knowledge for development of less harmful and more effective antimicrobial agents for treatment of urinary infections of urinary tract. The levels of the total phenolic content indicated that C. vulgaris extracts are rich source of the phenolic compounds.

TABLE 2
Antibacterial activities of aqueous, ethanol and ethyl acetate extracts from
leaves and flowers of C. vulgaris against tested strains of bacteria.

| | Aqueous extract | | Ethanol extract | | Ethyl acetate extract | | Amoxicillin | |
|---|---|---|---|---|---|---|---|---|
| Species | MIC¹ | MBC² | MIC | MBC | MIC | MBC | MIC | MBC |
| E. coli [symbol] | 20 | 40 | 20 | 40 | 40 | [symbol] 40 | 1000 | 2000 |
| E. coli [symbol] | 20 | 40 | 20 | 40 | 40 | >40 | 4000 | 8000 |
| E. coli [symbol] | 10 | 20 | 20 | 40 | 40 | [symbol] 40 | 4 | 8 |
| E. coli [symbol] | 20 | 40 | 20 | 40 | 40 | >40 | 2 | 4 |
| E. coli [symbol] | 10 | 20 | 20 | 40 | 40 | >40 | 2000 | 4000 |
| E. coli [symbol] | 10 | 20 | 20 | 40 | 40 | [symbol] 40 | 2000 | 4000 |
| E. coli [symbol] | 10 | 20 | 20 | 40 | 40 | [symbol] 40 | 4 | 4 |
| E. coli [symbol] | 10 | 20 | 20 | 40 | 40 | >40 | 1000 | 2000 |
| E. coli [symbol] | 10 | 20 | 20 | 40 | 40 | >40 | 4000 | 8000 |
| E. coli [symbol] | 10 | 20 | 20 | 40 | 40 | >40 | 1000 | 2000 |
| E. faecalis [symbol] | 20 | 40 | 20 | >40 | 40 | [symbol] 40 | 0.977 | 125 |
| E. faecalis [symbol] | 10 | 20 | 20 | >40 | 40 | >40 | 0.488 | >125 |
| E. faecalis [symbol] | 10 | 20 | 20 | >40 | 40 | >40 | 0.488 | [symbol] 125 |
| E. faecalis [symbol] | 10 | 20 | 20 | [symbol] 40 | 40 | >40 | 0.488 | >125 |
| E. faecalis [symbol] | 20 | 40 | 20 | >40 | 40 | [symbol] 40 | 0.488 | [symbol] 125 |
| E. faecalis [symbol] | 20 | 40 | 20 | [symbol] 40 | 40 | [symbol] 40 | 0.488 | [symbol] 125 |
| E. faecalis [symbol] | 10 | 20 | 20 | >40 | 40 | >40 | 0.977 | >125 |
| E. faecalis [symbol] | 10 | 20 | 20 | >40 | 40 | >40 | 0.488 | 125 |
| E. faecalis [symbol] | 10 | 20 | 20 | [symbol] 40 | 40 | >40 | 0.488 | >125 |
| E. faecalis [symbol] | 10 | 20 | 20 | [symbol] 40 | 40 | [symbol] 40 | 0.488 | [symbol] 125 |
| P. vulgaris [symbol] | 2.5 | 5 | 10 | 40 | 5 | 10 | [symbol] 4000 | [symbol] 4000 |
| P. vulgaris [symbol] | 2.5 | 5 | 10 | 40 | 5 | 20 | [symbol] 4000 | >4000 |
| P. vulgaris [symbol] | 2.5 | 5 | 10 | 40 | 5 | 10 | >4000 | >4000 |
| P. vulgaris [symbol] | 2.5 | 5 | 10 | 40 | 5 | 10 | 0.977 | [symbol] |
| P. vulgaris [symbol] | 2.5 | 5 | 10 | 40 | 5 | 20 | >4000 | [symbol] 4000 |
| P. vulgaris [symbol] | 2.5 | 5 | 10 | 40 | 5 | 20 | 0.977 | [symbol] |
| P. vulgaris [symbol] | 2.5 | 5 | 10 | 40 | 5 | 10 | >4000 | >4000 |
| P. vulgaris [symbol] | 2.5 | 5 | 10 | 40 | 5 | 20 | 2000 | >4000 |
| P. vulgaris [symbol] | 2.5 | 5 | 10 | 40 | 5 | 20 | >4000 | [symbol] 4000 |
| P. vulgaris [symbol] | 2.5 | 5 | 10 | 40 | 5 | 20 | >4000 | [symbol] 4000 |

¹Minimum inhibitory concentration (MIC) and
²minimum bactericidal concentration (MBC) values are given as mg/ml for plant extracts and μg/ml for antibiotic. [symbol]
indicates data missing or illegible when filed

### Example: Desert Harvest Calluna vulgaris Study Protocol

### Introduction

Urinary Tract Infections (UTIs): Urinary tract infections (UTIs) are one of the most frequent clinical bacterial infections in women, accounting for nearly 25% of all infections. Around 50-60% of women will develop UTIs in their lifetimes. Having frequent sexual intercourse is one of the greatest risk factors for RUTIs. In a subgroup of individuals with coexisting morbid conditions, complicated RUTIs can lead to upper tract infections or urosepsis. The standard initial treatment is antimicrobial therapy, but alternative preventative strategies are needed to reduce exposure to antibiotics.

Calluna vulgaris: Heather, (Calluna vulgaris L. Hull, fam. Ericaceae) is an evergreen shrub. C. vulgaris can be found in most parts of Europe and Northern America from lowland up to alpine regions. C. vulgaris has been used in ethnopharmacology as an antiseptic, antibacterial, cholagogue, diuretic, expectorant, antirheumatic and anti-inflammatory agent. The levels of the total phenolic content indicated that C. vulgaris extracts are rich source of the phenolic compounds. The result of recent research suggests that extract from C. vulgaris, tested in vitro, show great potential as a natural antibacterial agent. Thus, they may be useful in the treatment of infectious diseases caused by urinary tract pathogens, including Escherichia coli, Enterococcus faecalis and Proteus vulgaris. Extracts of Calluna vulgaris may have a significant effect on the prevention of the infections of urinary tract.

### Study Objectives

The primary objective of the study is to determine the most effective delivery method of Calluna vulgaris in capsule form versus tincture form for women with recurrent urinary tract infections. The symptoms that will be monitored will include urinary frequency, nocturia, dysuria, urgency, and suprapubic pain. Response to therapy will be monitored by the Recurrent Urinary Tract Infection Symptom Scale (RUTISS).

### Study Design

The study will be designed as a case-control trial with subjects randomized in a 1:1 fashion. Subjects will be females with at least who have had at least three episodes of a Urinary Tract Infection (UTI) in the past twelve months or two UTI episodes in the past 6 months. At least one of the UTI episodes must be associated with a positive urine culture with a traditional uropathogen including Escherichia coli, Enterococcus faecalis and Proteus vulgaris.

Each subject will be randomized to Calluna vulgaris capsules, a Calluna vulgaris tincture, or D-Mannose capsules with a dosing regime of one capsule per day or one dropper full per day. Each subject will take the study medication for twelve weeks and be monitored monthly.

Efficacy will be measured by the Recurrent Urinary Tract Infection Symptom Scale (RUTISS). Subjects must receive at least 80% of the study medication regime to be considered evaluable. All subjects who receive at least one dose of study medication will be evaluated for safety.

The study will be conducted with Live UTI Free volunteers with approximately thirty total subjects enrolled. It is anticipated that at least 15 of these subjects will be considered evaluable. Enrollment will take approximately one month with each subject's individual participation lasting three months. The principal investigator site will complete an Enrollment Log weekly during the enrollment period and email it to the attention of the Desert Harvest Research Coordinator study@desertharvest.com.

Dispensing and Collecting Study Medication: Each enrolled study participant will receive one bottle of either Calluna vulgaris capsules, a Calluna vulgaris tincture, or D-Mannose capsules once a month to their preferred address. Shipment tracking information will be provided to participants via email, with participants immediately notifying research coordinator of any delivery issues to maintain study compliance.

Non-Serious Adverse Events: Adverse events are defined as a new medical condition or worsening of an existing medical condition, not to include the disease being studied. Adverse events will be recorded on the Adverse Event Reporting Form. The research coordinator will make a determination of the event and its relationship to the study medication.

Serious Adverse Events: A serious adverse event is any adverse event occurring that results in any of the following: Death, A life-threatening event, Inpatient hospitalization or prolongation of existing hospitalization-hospitalization for an elective treatment of a pre-existing condition is not a serious event, A disability or incapacity, An important medical event which, though not included above, may jeopardize the subject and may require medical or surgical intervention.

In the event of a serious adverse event, the Research Coordinator at Desert Harvest must be notified within 24 hours of becoming aware of the event. All serious adverse events will be recorded on an Adverse Event Reporting Form.

Early Termination: Subjects will be withdrawn from the study after randomization for the following conditions: New onset of a urinary tract infection, Onset of any other infection requiring antibiotics, Onset of any condition requiring administration of NSAIDs or corticosteroids, Exacerbation of genital herpes, Onset of vaginitis, Significant change of concurrent medical conditions, Demonstrated noncompliance with study protocol, specifically dosing of study medication.

Subjects will be instructed to notify the study staff of any changes in their medical condition as soon as they occur. Early termination procedures will be completed as soon as possible.

### Subject Selection

### Inclusion Criteria:

Females, aged 18 years or older.

Participant must sign and date the informed consent.

Post-menopausal for a minimum of 1 year or negative pregnancy test if participant is of childbearing potential and agreement to acceptable contraceptive use for all three months of study if participant is sexually active. Medically acceptable methods of contraception include hormonal contraception (i.e. estrogen, and/or progesterone or preparations that contain a combination of these hormones), non-hormonal intrauterine device or double barrier method (i.e. condom with foam or vaginal spermicidal suppository, or diaphragm with spermicide), vasectomy of sole sexual partner, or complete abstinence.

Participants must have had at least three episodes of a Urinary Tract Infection (UTI) in the past twelve months or two UTI episodes in the past 6 months. At least one of the UTI episodes must be associated with a positive urine culture with a traditional uropathogen including Escherichia coli, Enterococcus faecalis and Proteus vulgaris.

### Exclusion Criteria

Known allergy or intolerance to Calluna vulgaris or Aloe Vera in any form as reported by the participant.

Active urinary tract infection at the time of trial inclusion. History of bladder tumors (malignant or benign).

History of urethral, uterine, cervical, or vaginal cancer within the previous five years.

Current active bladder or urethral calculus.

Current infection related to urinary lithiasis. Any immunological disease requiring active immunotherapy.

Any disease which, in the opinion of the investigator, may be unstable or have bearing on the outcome of the study, including severe debilitating concurrent medical conditions such as coronary artery disease, azotemia, moderate to severe hepatic insufficiency, etc.

Unlikely to be compliant due to unmanaged medical or psychological problem, including dementia, aphasia, or other deficits of cognition or speech/language function that will interfere with the participant's ability to complete the study.

Substance abuse or dependency problem within the past two years for which patient received no treatment.

Pregnant or lactating females.

### Quality Assurance

Desert Harvest (or its designee) will monitor the study remotely. A quality assurance check will be performed to ensure the participants are complying with the protocol. All records will be reviewed for completeness, legibility, and acceptability. At the completion of the study, Desert Harvest (or its designee) will archive all records and study materials. Copies of records will be retained at the site per individual site policy.

### REFERENCES

[13] Wootton-Beard C, Moran A, Ryan L. Stability of the Total Antioxidant Capacity and Total Polyphenol Content of 23 Commercially Available Vegetable Juices before and after in vitro Digestion Measured by FRAP, DPPH, ABTS and Folin-Ciocalteu Methods. Food Res Int 2011; 44 (1) 217-224. http//dx.doi. org/10.1016/j.foodres.2010.10.033.
[14] Quettier-Deleu C, Gressier B, Vasseur J, Dine T, Brunet C, Luyckx M, et al. Phenolic Compounds and Antioxidant Activities of Buckwheat (Fagopyrum esculentum Moench) Hulls and Flour. J Ethnopharmacol 2000; 7235-42. http//dx.doi.org/10.1016/S0378-8741 (00) 00196-3.
[15] Farrell D J, Morrissey I, Rubeis D D, Robbins M, Felmingham D. A UK multicentre study of the antimicrobial susceptibility of bac-terial pathogens causing urinary tract infection. J Infection 2003; 46 (2) 94-100. http//dx.doi.org/10.1053/jinf.2002.1091.
[16] NCCLS (National Committee for Clinical Laboratory Standards). Methods for Dilution Antimicrobial Susceptibility Tests for Bacteria That Grow Aerobically. Approved Standard M7-A4 Wayne P A 1997.
[17] Xia D, Wu X, Shi J, Yang Q, Zhang Y. Phenolic compounds from the edible seeds extract of Chinese Mei (Prunus mume Sieb. Et Zucc) and their antimicrobial activity. Lwt-Food Sci Technol 2011; 44 (1) 347-349. http//dx.doi.org/10.1016/j.lwt.2010.05.017.
[18] Qader S W, Abdulla M A, Chua L S, Najim N, Zain M, Hamdan S. Antioxidant, Total Phenolic Content and Cytotoxicity Evaluation of Selected Malaysian Plants. Molecules 2011; 16 (4) 3433-3443. http//dx.doi.org/10.3390/molecules16043433.
[19] Dashputre N L, Naikwade N S. Evaluation of Anti-Ulcer Activity of Methanolic Extract of Abutilon indicum Linn leaves in experimen-tal rats. Int J Pharm Sci Drug Res 2011; 3 (2) 97-100.
[20] Muthuraman A, Sood S, Singla S K. The antiinflammatory potential of phenolic compounds from Emblica officinalis L. in rat. Inflammopharmacology 2011; 19 (6) 327-334. http//dx.doi. org/10.1007/s10787-010-0041-9.
[21] Özçelik B, Kartal M, Orhan I. Cytotoxicity, antiviral and antimicro-bial activities of alkaloids, flavonoids, and phenolic acids. Pharm Biol 2011; 49 (4) 396-402. http//dx.doi.org/10.3109/13880209.2010.519390.
[22] Gordien Y, Gray A I, Ingleby K, Franzblau G, Seidel V. Activity of Scottish plant, lichen and fungal endophyte extracts against Mycobacterium aurum and Mycobacterium tuberculosis. Phytother Res 2010; 24 (5) 692-698.
[23] Huttunen S, Riihinen K, Kauhanen J, Tikkanen-Kaukanen C. Antimicrobial activity of different Finnish monofloral honeys against human pathogenic bacteria. APMIS 2013; 121 (9) 827-834. http//dx.doi.org/10.1111/apm.12039.
[24] Allen K L, Molan P C, Reid G M. A survey of the antibacterial activity of some New Zealand honeys. J Pharm Pharmacol 1991; 43 (12) 817-822. http//dx.doi.org/10.1111/j.2042-7158.1991.tb03186.x.
[25] Vučić D M, Petković M R, Rodić-Grabovac B B, Stefanović O D, Vasić S M, Čomić LjR. Phenolic content, antibacterial and anti-oxidant activities of Erica herbacea L. Acta Pol Pharm 2013; 7 (45) 5130-5136.
[26] Vučić D M, Petković M R, Rodić-Grabovac B B, Stefanović OD, Vasić S M, Čomić LjR. Antibacterial and antioxidant activities of bilberry (Vaccinium myrtillus L.) in vitro. Afr J Microbiol Res 2013; 7 (45) 5130-5136.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of treating a Urinary Tract Infection in a subject, comprising: administering an effective amount of Calluna vulgaris (C. vulgaris) to the subject.

2. The method of claim 1, wherein phenolic content of Calluna vulgaris is between about 67.55 mg to about 142.46 mg of GAE/g of Calluna vulgaris and an effective amount of flavonoid content between about 42.11 to about 63.68 mg RUE/g of Calluna vulgaris.

3. The method of claim 2, wherein the Calluna vulgaris comprises an antibacterial activity between about 2.5 Minimum inhibitory concentration (MIC) and 40 MIC and an antibacterial activity between about 2.5 minimum bactericidal concentration (MBC) and about 40 MBC.

4. The method of claim 3, wherein the Calluna vulgaris is between about 0.1 mg/ml to about 40 mg/ml of Calluna vulgaris.

5. The method of claim 4, wherein Calluna vulgaris is in a powder form or a tincture form.

6. A composition or formulation for treating a Urinary Tract Infection, comprising an active ingredient or pharmaceutical composition from Calluna vulgaris (C. vulgaris) for the prevention and treatment of Urinary Tract Infections.

7. The composition of claim 6, wherein phenolic content of Calluna vulgaris is between about 67.55 mg to about 142.46 mg of GAE/g of Calluna vulgaris and an effective amount of flavonoid content between about 42.11 to about 63.68 mg RUE/g of Calluna Vulgaris.

8. The composition of claim 7, wherein the Calluna vulgaris comprises an antibacterial activity between about 2.5 Minimum inhibitory concentration (MIC) and 40 MIC and an antibacterial activity between about 2.5 minimum bactericidal concentration (MBC) and about 40 MBC.

9. The composition of claim 8, wherein the Calluna vulgaris is between about 0.1 mg/ml to about 40 mg/ml of Calluna vulgaris.

10. The composition of claim 9, wherein the composition or formulation from Calluna vulgaris (Heather) plant is in powder or tincture form.
